(19) United States
(12) Patent Application Publication
(10) Pub. No.: US 2023/0001032 A1
(43) Pub. Date: Jan. 5, 2023

(54) DISINFECTING SYSTEM FOR FOOD PROCESSING EQUIPMENT

(71) Applicant: D-TECH EHF, Reykjavik (IS)

(72) Inventor: Ragnar OLAFSSON, Reykjavik (IS)

(73) Assignee: D-TECH EHF, Reykjavik (IS)

(21) Appl. No.: 17/756,029

(22) PCT Filed: Nov. 11, 2020
(86) PCT No.: PCT/IS2020/000002

§ 371 Date: May 13, 2022

(30) Foreign Application Priority Data
Nov. 13, 2019 (IS) 9131

(51) Int. Cl. A61L 2/22; A61L 2/24

(52) U.S. Cl. CPC A61L 2/22; A61L 2/24; A61L 2202/15

(57) ABSTRACT

A system and method for disinfection of food processing equipment is described. The system comprises one or more nozzle for delivering disinfecting fog, and is adapted to be used so that there is free space in front of the nozzle, and an angle of at least 20° for the expanding fog. Further disclosed is a food processing system comprising at least one food processing unit that comprises a housing and food processing means within the housing, the housing being provided with a plurality of nozzles on an inside surface to deliver disinfectant and/or cleaning fluid towards the food processing means. Methods of disinfecting food processing equipment are also provided.

### RELATED APPLICATIONS

This application is the U.S. National Phase of and claims priority to International Patent Application No. PCT/IS2020/000002, International Filing Date Nov. 11, 2020, entitled Disinfecting System For Food Processing Equipment; which claims benefit of and priority to Iceland Application No. 9131 filed Nov. 13, 2019 entitled Disinfecting System For Food Processing Equipment; both of which are incorporated herein by reference in their entireties.

### FIELD

The invention relates to disinfecting systems and methods for using such systems, in particular systems and methods for disinfecting food processing equipment.

### INTRODUCTION

The fish and meat processing industry is highly sensitive to infections and must be operated under strict cleanliness and the best possible hygienic conditions.

Although the raw material, such as fish, meat or poultry, when being cut or trimmed is usually kept cold and may be cold, such as below 4° C. when entering the process going into the cutting machines, the surrounding temperature in the operation facilities, is usually much higher, or often 12-15° C., as this is the normal lower limit of comfort temperature for workers, and people should not be working for extended time in rooms at temperature below 12° C.

Since the cutting machines are mainly of metal they conduct heat very efficiently and in addition complex mechanics inside cutting machines also create a lot of heat and therefore the temperature inside the machines will be even higher than the surrounding room temperatures. Temperature inside commercial cutters, such as in the FleXicut system from Marel, when processing fish, have been measured to be above 18° C. when the outside room temperature was about 14° C. Therefore much higher temperatures are observed inside the cutters than the product should ever experience according to guidelines and regulations, and therefore, even though the process is very rapid, the temperature of the product is likely to increase during the process.

But the main problem here may not be the relatively small increase in the product temperature, but since the whole environment inside the cutter has high humidity and is loaded with nutritious debris from the food, it makes a very favorable growth conditions for all kinds of harmful bacteria. Few pathogenic bacteria can grow and multiply at temperatures below 4° C., but dangerous bacteria such as Listeria can grow at that temperature and very rapidly if temperature has reached 8° C. or more (Pradhan et al. 2012), Moeretroe et al. 1999). Other highly pathogenic bacteria, such as Salmonella (Morey and Singh, 2012) and enterohemorrhagic E. coli O157:H7 (Huang, 2010) have been shown to grow even at 8° C. and to multiply quite rapidly at temperatures above 15° C.

Modern fish and meat cutting and trimming machines are very high-tech, computer operated robots with cutting and picking done by high-speed knives, or beams of laser, water or gas. This includes various sensors, such as high-resolution cameras allowing 3D vison and very high cutting frequency of thousand strokes per minute, which can be reached by extremely fine-tuned systems. By using the most precise servo-drivers and fastest processors, operated by advanced software, extremely high degree of cutting precision and speed performance is obtained. Different versions and applications of such cutting and trimming machines are well known in the art, including examples of such machines as described in patents or patent applications by Hjalmarsson and Jonsson, (2010), Finnsson and Hallvardsson (2014), Hallvardsson and Gudlaugsson (2014) and Blaine et al. (2016).

This requires that highly sensitive apparatus needs to be included inside or as part of otherwise rugged and robust apparatus normally used in fish and meat processing. This has several consequences such as that during operation the processing machines must be closed, but the high-power mechanical operation inside the machines create very fine droplets and particles of flesh that will circulate in the air inside the apparatus. Also due to the very fine and sticky nature of the material, it can reach all corners and crevices and adhere to surfaces inside and throughout the machinery. As this material is also an excellent nutrition for the growth of bacteria, the whole situation becomes very sensitive. Therefor a thorough and frequent cleaning and disinfection is of outmost importance in the operation.

The producers of such equipment, such as Marel (https://marel.com/products-solutions/flexicut/), Valka (https://valka.is/valka-to-debut-cutter-for-pre-rigor-salmon-at-spg-in-brussels/) and Scanvaegt, (https://www.scanvaegt.com/en/food-industry/news/iffa-world-premiere/) typically claim that their systems are made of a very sturdy, yet simple outside design with smooth surfaces which minimizes the risk of residue sticking inside the machine. They also typically claim that motors are enclosed in hermetically sealed cabinets in a standard waterproof environment, and that all metal-surfaces and plastic parts are with smooth surface and approved for food contact to minimize any residues sticking to the surfaces. The producers also prepare the machines for easy cleaning, such as that the entire cabinets will easily open up, often top-hinged and can be tipped upwards, allowing free access to the inside of the machine. The open construction of conveyor belts and other mechanical parts provide complete access, and also many components, such as cutting units and knives can be easily removed or tipped up or down for better cleaning access. This is to secure the best possible access to all parts, and therefore allowing the interior of the machine to be thoroughly hosed down. This hose-down cleaning is typically done with high-pressure water jets in order to effectively remove any attached debris from even the most difficult-to-reach parts and crevices of the apparatus.

This cleaning practice of high pressure hosing has however, led to frequent damage or skewing of the highly sensitive and precise equipment, resulting in frequent, expensive stops and repairs, or problematic function and bad cuts which lowers the productivity and yield of the fish or meat products.

Most cleaning and disinfection work are normally performed on night shifts and often under relatively unsafe conditions. The cleaning workers typically perform their cleaning operations with little training and supervision by senior management.

Therefore this has led to that many fish and meat producers have stopped using the high-pressure water jets for cleaning, to prevent damage and use instead only low-pressure water spraying for cleaning, which again is not as effective in removing all the attached debris and bacterial biofilm. This also results in longer cleaning time and higher salary costs.

A combination of all these factors discussed above has therefore led to insufficient cleaning, resulting in serious hygienic problems in many fish and meat processing factories. The poor cleaning and high temperatures during operation has led to serious bacterial problems, since even if the bacteria are not growing in the products themselves, they are growing in the surroundings and get transferred over to the products, which then get infected and become bacteria-positive in regular quality checks. This then results in higher frequency of customer rejects due to unacceptably high bacterial counts in the finished products.

In some food processing applications (e.g., fish, meat and poultry), the amount of fine debris of protein and fat has a strong tendency to stick to surfaces, leading to rapid buildups of biofilms. It is therefore difficult and requires the use of strong chemicals to clean surfaces of clinging biofilms, fat and proteins. Therefor much human contact towards such chemicals should be avoided and automatic cleaning and disinfection process which minimizes the amount of chemicals used in the process is highly desirable.

As part of the processing of food components of animal origin as described here, the processing often ends with that the whole animal carcass, or more often some parts thereof, are frozen before storage. The freezing is normally done in so-called blast freezers, with very low temperatures, but also with very high wind or blasting air. This requires that powerful freezing units with strong fans are necessary part of such blast freezers. This means that air is circulating through the freezer units and fine dust of flesh is created and tends to build up on the freezer elements, fan blades and other surfaces. This can have insulating effect on the freezer elements and decrease the freezing effect. So even when no bacteria are growing at the low operating temperatures of such blast freezers, they need to be cleaned and then the temperature rises into the growth-temperature range of many bacteria. Due to the complex and compact structure of the blast blowers they again are difficult to clean and disinfect. It has frequently been observed by many food processors that the first few lots of frozen products coming out of such blast freezers after cleaning, that they have unacceptably high bacterial counts.

During the process of cleaning and disinfection, conveyors, blast freezers or other equipment should be run at slow speeds to ensure that all surfaces are contacted. The modern complex, high tech food processing machines must be closed when being run for safety reasons, and which therefore makes the manual cleaning even more difficult. Normally employees should not be allowed to go inside the machines while operating, as this is not only dangerous, but also their clothes, gloves and booths may be a source of contamination that can be brought into the equipment.

Disinfectants are commonly applied as fogs in the chilled food industry (Burfoot et al. 1999). Recent research has shown that fogging is effective in reducing the number of organisms on upward facing surfaces but, in general, is less effective on vertical or downward-facing surfaces. Fogging also reduces the number of viable airborne organisms. Numerical models of the dispersion of airborne particles have been used to simulate the fogging process. These models, with supporting experiments, showed that fogs should be most effective when the median diameter of the fog droplets lies between 5 and 20 μm. Droplets in this size range disperse well and settle within few minutes. This gives good coverage in rooms and the fog clears from the air quickly enough not to pose disruption to factory operations (Crosfield et al. 2009).

Although rotating spray nozzles are well known and widely used for cleaning and washing inside many food containers, the use of fixed, high pressure nozzles for fog disinfection is much less known and still not widely used in many food processing operations.

The use of fog systems for disinfection of many different kind of facilities, rooms, pipes and tanks is well known in the art, as described by Chang and Chan-Myers (2012) and by Agmont E Silva, (2017). Such fog disinfection systems are known to be very economical and efficient, compared to direct wash or other disinfecting solutions, but specialized solutions for modern food processing equipment such as high-tech cutters and trimmers have not been available.

Therefor an improved cleaning and disinfection procedure for the satisfactory operation of the modern high-tech fish and meat cutters, as well as many associated parts such as vacuum cyclone, or blast freezers, is desperately needed.

### SUMMARY

The present invention relates to a system and method for disinfection of compartments that are designed and suitable for food processing equipment, such as equipment for high precision cutting and trimming of food products.

The invention also relates to a system comprising one or more food processing units that have internal disinfecting and/or cleaning means, such that the food processing units can be disinfected and/or cleaned without attaching or using external cleaning means.

Accordingly, in an aspect, the invention provides a system for the disinfection of food processing equipment. The food processing equipment preferably comprises means for input, processing and/or output of food parts. The system can preferably contain one or more nozzle for delivering disinfecting fog; piping means for delivering gas and disinfectant liquid from respective sources of gas and disinfectant liquid to the one or more nozzle; and at least one gas flow rate controller and at least one liquid flow controller, for controlling gas and liquid flow rates in the one or more nozzle.

The nozzle can be adapted to be positioned on food processing equipment so that during use, disinfecting fog delivered by the at least one nozzle is directed towards at least one internal surface of the food processing equipment, wherein free unobstructed space in front of the nozzle, for the expansion of the sanitizing fog towards the at least one internal surface, is at least 20 cm.

Furthermore, the nozzle can be adapted to deliver sanitizing fog that expands from the nozzle at an angle of at least 20° relative to the direction of the expansion.

In the present context the term “fog” is intended to mean a cloud or stream of small water droplets in an atmosphere, which typically is ambient atmosphere (i.e., air). The water droplets in the fog, as described herein, are typically smaller than about 20 μm, and can be even smaller, such as less than about 10 μm or less than about 5 μm.

Nozzles for generating fog are known in the art. The nozzles can be any suitable such nozzle, for example high-pressure nozzles. Nozzles typically have an inlet for liquid, an inlet for pressurized gas and a small outlet orifice. The droplets are formed inside the nozzle where the pressurized gas meets incoming disinfectant liquid and is released through the outlet orifice.

The system can be adapted to provide appropriate flow rates depending on the intended use. Thus, in the case where a large volume of space needs to be provided with a disinfecting fog, a high flow rate through the nozzle is appropriate. Conversely, for smaller confined spaces, an appropriately smaller flow rate may be appropriate. In general, the appropriate flow rate of liquid in the nozzle can be in the range of about 30 to 500 mL per minute. The operating pressure can generally be in the range of about 0.5 to about 5 bar.

Gas flow rate in the nozzle can generally be between 10 to 100 L per min at operating pressures between 3 and 7 bar. The combination of gas and liquid flow rates can be adjusted to achieve the required size of water droplets and/or delivery rate, as known in the art.

The system can comprise one or more disinfectant and/or detergent supply. The system can be adapted to mix disinfectants/detergents from two or more supplies prior to delivery to the nozzles, optionally also from a water supply, to mix water into the disinfectants. Thereby, various mixes or concentrations of disinfectants or detergents to be supplied to the one or more nozzle are provided.

The system can comprise a housing positioned near, or at some distance from the food processing equipment and can be opened for servicing. The system housing is independent from the food processing equipment and can contain all needed operational units with control panel, pressure pump and equalizer and respective liquid containers.

The system can be made to fit into a small space and can be separate from or attached to the interior of food processing equipment, such as a high precision cutting and trimming machines. The system can be operated via electronic and remote connection. Therefor the operator does not need to open the machine for the disinfection to take place. The system can comprise a reporting function, to keep track of and report progress of disinfecting operations by the system.

In another aspect, the invention provides a food processing system having internal disinfecting and/or cleaning means, the system comprising at least one food processing unit, wherein the at least one food processing unit comprises a housing and food processing means arranged within said housing and a plurality of nozzles arranged on an inside surface of said housing, the nozzles being adapted to deliver a stream of a disinfectant and/or cleaning fluid towards the food processing means. The nozzles can be arranged on an internal surface of the housing such that there is unobstructed space between the nozzles and food processing means within said housing, in the direction of delivery of a stream of disinfectant from the nozzles. Preferably the unobstructed space, is at least 10 cm, at least 20 cm or at least 30 cm. The nozzles can be provided so as to extend through the housing, to deliver disinfectant and/or cleaning solutions in the form of a fog from an outside source towards internal food processing equipment.

There can be piping means to deliver disinfectant/detergent from respective supplies to the nozzles. Piping means can be any means known in the art for delivering liquid solutions and air, including but not limited to metal, composite material and plastic pipes or tubing.

Nozzles can be permanently affixed to the food processing equipment. The nozzles can also be positioned temporarily to the equipment, e.g. for the purpose of disinfection and/or cleaning. The food processing equipment can be provided with securing means, for attaching and/or securing the nozzles to the equipment during such use. There can be complementary fastening means on the nozzles or attached to the nozzles. Thus, during use the nozzles can be engaged with the fastening/securing means on the food processing equipment.

For effective disinfection to take place in a given unit of a food processing equipment a space of up to 1 m³ can be serviced by one nozzle when producing suitable fog for 1-2 minutes. This requires that the space is largely unobstructed, or if partly obstructed to the extent that the obstruction covers not more than half of the area facing the direction of the disinfecting fog. If the obstruction covers more than half of the area than one or more additional nozzles must be placed inside the given space. If the given space of no more than 1 m³ is unobstructed a single nozzle can effectively spray the disinfecting fog for up to 2 m distance from the servicing nozzle. Usually a food processing operation such as slaughtering, cutting, removal of intestines, cleaning, cooling and packing, are performed in separate units or spaces, such that each unit or space must be disinfected and therefore one or more nozzles must be placed in each unit.

There can thus in some embodiments be at least one nozzle for providing disinfecting fog for each m3 of space that needs to be serviced/disinfected. In some embodiments, there can be a higher density of nozzles, such as one for each 0.7 m³, one for each 0.5 m³, one for each 0.3 m³ of space to be serviced/disinfected.

The steps involving the first cutting and removal of intestines from the carcass of fish or poultry are especially sensitive since the intestines contain high numbers of both food-spoiling and disease-causing bacteria. Some bacteria that can cause diseases in humans are common part of the normal flora of the environment or in animal intestines, such is the case for Listeria in fish and Campylobacter in chickens. Listeria is especially difficult for the fish-processing industry, since it can survive and grow at low temperature and is known to be persistent in some fish-processing facilities. Once Listeria has infected some food-processing equipment it is difficult to get rid of and can continue to spread and infect other parts of the food processing operation.

Also provided is method of disinfecting food processing equipment having a housing and internal means for input, processing and/or output of food parts comprised within said housing, the method comprising introducing a disinfecting fog containing droplets into the food processing equipment, so as to flood internal surfaces of the food processing equipment with the disinfectant, wherein the introducing is performed by delivering disinfecting fog via one or more nozzle that is positioned on an internal surface of the housing. The method can preferably be performed using a system as disclosed herein, or within a food processing system having internal disinfecting and/or cleaning means, as disclosed herein.

There can be an optional pre-rinsing step that precedes the disinfecting step, wherein a fog containing only water droplets are introduced, to prewet the equipment/system to be disinfected.

There can also, or alternatively, be a post-washing step that follows the disinfection step, wherein also fog containing only water droplets are introduced, to wash the equipment/system that has been disinfected, thereby removing excess disinfectant.

Disinfection normally takes place in a sequence of steps which are required to be carefully followed for an effective disinfection to take place. A typical disinfection can involve the following steps and sequence.

1. Spraying of water fog, by using only pressurized air and water lines, i.e. a step of introducing a fog containing aqueous droplets containing water only, and do not contain disinfectant. This can be done for 1-2 minutes but the time can in general be set from 1 to 99 min. This water fog step is important as it wets the surfaces to be disinfected and increases air humidity, resulting in longer lasting fog in the next step such that the disinfectant will better cover the whole volume and it results in improved disinfection.
2. Immediately following is the disinfection step that includes the spraying of fog made from the appropriate disinfection mixture, by using one or more chemical mixtures diluted in water and the pressurized air. This normally is for 5-10 minutes but time can be set from 1 to 99 min.
3. Immediately following the disinfection step where step 1 above is repeated by spraying water fog by using only pressurized air and water lines. This normally is for 1-3 minutes but time can be set from 1 to 99 min. This rinsing is required by authorities since all chemicals that are used during disinfection, must be removed after disinfection and before any food processing takes place.
4. Immediately following the rinse or cleaning step the nozzle lines are blown with just pressurized air to remove all water or chemicals from inside the systems in order to prevent possible formation of ice, deposits, or corrosion inside the system. This normally is for 1-2 minutes but time can be set from 1 to 99 min.

Disinfectants compatible with the systems and methods of the invention can in general be any disinfectants known in the art. For example, the disinfectant can be comprised of, or contain, one or more liquid disinfectant that is aqueous, or substantially aqueous. The liquid disinfectant can contain one or more different oxidative-process type disinfectants, comprising a plurality of different active substances. The liquid disinfectant can be made from aqueous solutions containing different concentrations of dissolved materials made of different active disinfectant substances well known in the art and composed of a mixture of active substances and liquid of different proportions.

Useful disinfectant include, but are not limited to disinfectants that include one or more active substances selected from isopropyl alcohol and dodecyldimethylammonium chloride, benzalkonium chloride, and Poly(hexamethylene biguanide) hydrochloride.

The disinfectant may further include one or more additional active disinfecting substances such as Peroxyacetic acid, Peracetic acid and Ethaneperoxoic acid.

The disinfectant may be provided as a liquid with a concentration of active disinfectants in the disinfectant liquid is 1-50% (w/w). It may be convenient to provide the disinfectant as a concentrate that is premixed with water or another appropriate solvent. Accordingly, the system may comprise a mixing unit, for generating a ready-to-use liquid disinfectant by mixing water or another appropriate solvent and one or more concentrated disinfectant solution.

The system of the invention can furthermore contain other components such that the disinfection time can be controlled. The system can thus contain one or more timer for controlling the time of any step in the disinfection process.

The invention furthermore contains components that allow remote electronic control and reporting to and from the system or any individual components of the system.

The system of the invention can be made by using any number of different materials available to those skilled in the art, or combination of different types of materials. Such materials include, but are not limited to, metals and various synthetic materials such plastics.

In certain embodiments of the invention the system can be controlled via a preprogrammed computer and software or as a coordinated task involving valves and switches coordinated beforehand and running in a prefixed/preprogrammed manner. Likewise, the measurement of temperature, levels of water and active substances for disinfection can be based on preprogrammed measurements in the system or through exact measurements in each unit and pipelines leading to the unit. Such measurements of the results of the disinfection and operation can be fed back to the control of the system to intensify and increase/decrease concentration of active substance in the fog, the droplet size of the fog, frequency of the delivery as well as length or volume of the treatment.

In one aspect of the invention, the disinfection is delivered through piping means (pipelines) that carry liquid and gas or air to the food processing equipment. As disclosed herein, the term “piping means” is intended to refer to any length of a tube or pipe that is suitable for carrying water and/or liquid. The piping means can be made from any suitable material, such as metal, alloy, plastic, rubber or the like that is known in the art. The piping means serve the purpose of delivering disinfectant liquid and gas (such as air) from respective sources of disinfectant liquid and gas/air to the nozzles.

In one embodiment, the invention is a method for controlled pre disinfection or pre cleaning of the food processing equipment before entering of foodstuffs into the equipment. Thus, it may be convenient to use the system disclosed herein to preclean or pre-disinfect a food processing equipment prior to use, i.e. prior to the processing of foodstuff into the equipment. Such precleaning or pre-disinfection can comprise flooding with water only, flooding with a cleaning solution, or flooding with disinfectant. If flooding with a disinfectant, there can be a pre-rinsing step and a post-rinsing step, to remove any excess disinfectant.

The invention uses known chemical and physical principles to combine in a new way a set of actions and design of a system that makes the actions possible and therefore useful and commercially viable. The invention is therefore an efficient method and is expected to be useful and consequently to have large potential for industrial application and commercial use.

Additional features and advantages of the present invention are described in, and will be apparent from, the following detailed description of the invention and the figures.

### BRIEF DESCRIPTION OF THE DRAWINGS

FIG. 1. Schematic drawing showing how food processing equipment with a disinfectant system in accordance with the invention.

FIG. 2. Schematic drawing showing how a second embodiment of a food processing equipment with a disinfectant system in accordance with the invention.

FIGS. 3A, 3B and 3C show disinfectant nozzles (A, B) that can be used with the invention and an illustration of the generation and spread of disinfectant fog from the nozzles (C).

FIG. 4 shows food processing systems that have been adapted to include internal disinfectant means in accordance with the invention.

FIG. 5. Shows the control and mixing unit that operates the disinfection procedures through a computer or remote control. Square boxes indicate solenoid valves and the round ball indicates the liquid pump. These are remotely and computer controlled during disinfection.

### DESCRIPTION

As described herein, the invention is used for rapid disinfection of cutting and trimming machines made for high-speed processing of meat or fish. The invention therefore makes possible quick and efficient disinfection resulting in elimination of damaging or pathogenic microorganisms that otherwise could render the products unsuitable for human consumption. The preferred embodiments of the invention adapted for disinfecting food products for extended time, will now be described in detail with reference to the drawings and figures provided.

Unless defined otherwise, all technical terms used herein have the same meaning as commonly understood by one of ordinary skill in the art to which the invention belongs. Although any methods and materials similar or equivalent to those described herein can be used in the practice or testing of the present invention, exemplary methods and materials are now described.

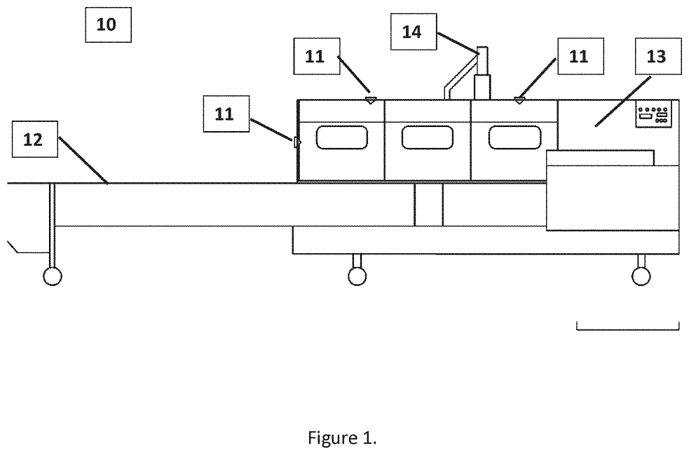

FIG. 1 shows a drawing of food processing equipment 10, indicating how nozzles can be positioned without disturbing the operation of the equipment, while at the same time providing for a complete loading of disinfecting fog when applied as described herein. The drawing can exemplify numerous types of food processing equipment, the internal machinery (not shown) being varied depending on the material source (fish, fish type, poultry, meat, meat cut, etc.).

The food processing equipment 10 has an inlet 12 for introducing food items to be processed into a housing 13 that contains food processing machinery, for example high precision cutting or trimming equipment. The inlet 12 is typically a conveyor belt as is known in the art.

Nozzles 11 are shown to be positioned on the side and top of the housing 13. The nozzles can be permanently positioned on the housing, i.e. the nozzles can be integral to the housing. The nozzles can also be removable from the housing, so that the nozzles are positioned as required on the housing for disinfection and removed after the disinfection has been completed.

There can therefore be openings or holes on the housing, for attaching and securing the nozzles on the housing and allow for the introduction of disinfectant fog into the interior of the housing. Piping means (not shown) provide disinfectant liquid and pressurized air to each of the nozzles 11.

The food processing equipment 10 can be a fish gutting machine, for example a salmon gutting machine. This machine uses vacuum to remove guts from salmon. Salmon is put inside the machine through inlet 12, which later cuts it open. Then a vacuum tube 14 is lowered into the equipment housing 13 and negative pressure air removes all guts into a cyclone where it is processed further. Vacuum is created by vacuum pump (not shown).

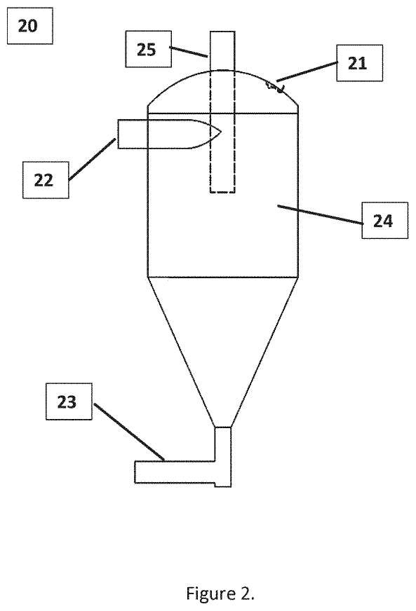

Fish guts are separated in a cyclone 20 as shown in FIG. 2. A high-speed rotating airflow is established within a cylindrical or conical container called a cyclone. Larger particles (fish guts) in the rotating stream have too much inertia to follow the tight curve of the stream, and thus strike the outside wall, then fall to the bottom of the cyclone where they can be removed. A similar unit is applied for removing poultry intestines.

This working principle creates perfect environment for Listeria to grow and contaminate other equipment. Listeria is very persistent and very hard to clean. It is very easy to spread into other places, while doing manual cleaning. The safest way to stop cross contamination and spreading is to clean automatically, without physically getting in the contact with contaminated area.

The cyclone 20 has an inlet 22 and an outlet 23, from which large pieces of gut are removed. A vacuum tube 25 extends into the cyclone, removing smaller food pieces and debris. A nozzle 21 is shown on an upper surface of the cyclone housing 24. To disinfect the cyclone between uses, disinfection cycles are generated through the nozzle 21. Additional nozzles can be provided as needed depending on the dimensions of the cyclone 20, preferably through the ceiling of the housing 24. However, nozzles can also be provided on side surfaces of the cyclone housing.

The nozzles 21 can be integral to the housing, or they can be attached to the housing for disinfection/cleaning, and subsequently removed.

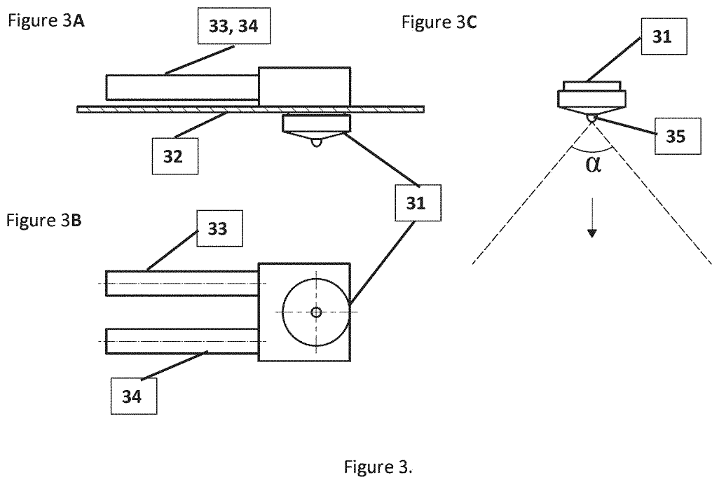

Turning to FIG. 3A there is shown a side view of a nozzle 31 that is attached to a panel 32 of a housing. The nozzle extends through the panel 32 of the housing, with piping 33, 34 providing disinfectant liquid and air or gas into the nozzle.

The nozzle can be integral to the housing, or it can be attached to the housing for use. In the latter case, it is preferable to close the housing by means of a plug or the like (not shown), to prevent contamination into or from the housing.

In FIG. 3B there is shown a bottom view of a nozzle 31. Piping 33, 34 provides flow of respectively disinfectant and gas into the nozzle 31. The flow is pressurized by respective pumps (not shown) and regulated by means of automated regulators, to provide an appropriate flow of disinfectant fog into the volume of space that is need of disinfection.

In FIG. 3C, a schematic view of a nozzle 31 is shown. The nozzle as an outlet orifice 35, through which fog expands into the open space to the disinfected. Due to the pressurization and the narrow opening on the nozzle, the fog expands in a cone-like manner, as illustrated by the dashed lines. The general direction of expansion is indicated by the arrow. The angle of expansion a indicates the spread of the cone-shaped stream of fog at the exit through the orifice 35. This angle is preferably at least 20°, but can be substantially greater, such as at least 30°, at least 40°, at least 50°, at least 60°, at least 70°, or at least 80°.

As the fog expands from the nozzle 31, the flow becomes increasingly chaotic, as the small droplets lose momentum as they travel through air. In a typical application, the fog from a nozzle can reach a distance of approximately 2 meters. However, as will be appreciated by the skilled person, this distance can be adjusted depending on the need, by varying the pressure and flow rate through the nozzle, and the style (shape and internal diameter of the orifice 35) of the nozzle 31. Thus, for certain applications, where hard-to-reach spaces need to be disinfected, it may be appropriate to use a relatively high pressure/flow rate and a relatively small angle of expansion.

For obstacles, i.e. internal machinery within a housing that is to be disinfected, that take up a large proportion of the space, it may be necessary to position additional nozzles on the housing, for example on either side of the machinery, above the machinery, as deemed appropriate to obtain adequate coverage of fog throughout the space, i.e. fog that surrounds internal surfaces on the machinery that are in need of disinfection.

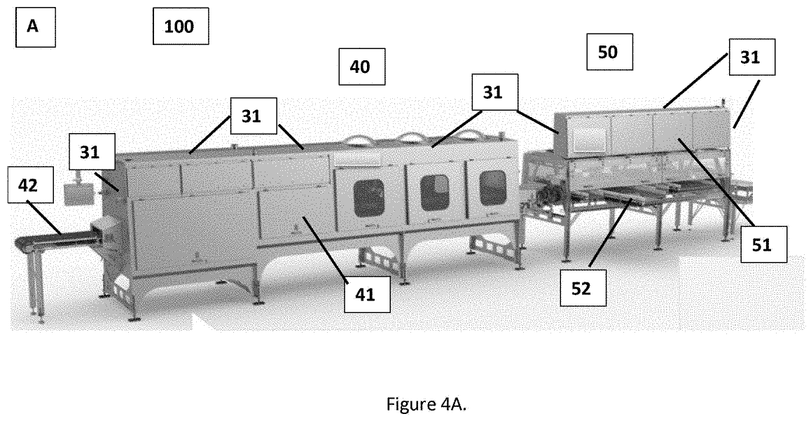

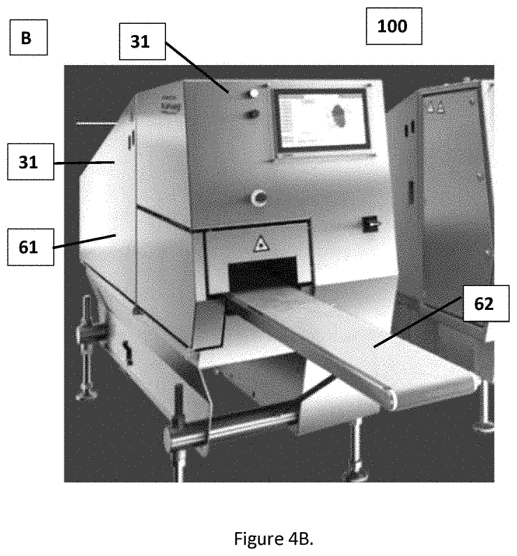

Turning to FIG. 4A there is shown a food processing system 100. The system comprises two food processing units 40, 50 that may be interconnected, for example by conveyor belts. The processing units may also be separate, i.e. lacking direct connecting or conveying means.

The food processing units consist of a housing 41,51, and internal food processing machinery (not shown), for example machinery for cutting or trimming food items. The food processing units each have inlets 42, 52, through which food to be processed enters, and outlets (not shown) through which the processed food exits.

On each food processing unit there can be provided nozzles 31, the exemplary position of which are indicated. The nozzles 31 can be provided on the sides or top of the housing 41, 51 as appropriate depending on the configuration of the internal processing machinery. Nozzles can be provided on the sides and/or roof of the housing as needed to be able to generate internal disinfectant fog that is sufficiently dense and sufficiently distributed to provide disinfection of internal surfaces.

An alternative food processing system 100 is shown in FIG. 4B, the system comprising a housing 61, an inlet conveyor belt 62, and internal food processing machinery (not shown). Nozzles 31 are indicated on the side wall and ceiling of the housing, to provide and stream of fog into the housing.

It will be appreciated that the types of nozzles 31, the placement and geometrical density of nozzles 31 on the food processing units and the selection of disinfectant and its concentration can be varied to achieve the required disinfection capability. Thus, an advantage of the invention is that the selection and placement of the nozzles can be changed depending on the functional requirement, i.e. the types, bulkiness and density of internal machinery that needs disinfection.

It can be convenient to operate the disinfecting system in three concrete steps, using the same nozzles and piping to deliver liquid and gas (air) into the nozzles.

In a first step, pressurized water and air are used to generate a pure water fog within the housing on which the nozzles are arranged. This first step serves the purpose of increasing the humidity of the space, thereby preventing evaporation during treatment with disinfectant fog. This first step can be performed over any suitable time period, e.g. from about 1 to about 100 minutes, as deemed appropriate.

In a second step, that typically will immediately follow the first step, disinfectant fog is delivered through the nozzles to provide dense disinfecting fog within the housing. This step can preferably be done over a relatively short period of time, to minimize the use of disinfectant. The disinfecting step can thus be performed over a period of about 1-10 minutes, such as about 1-5 minutes, about 1-4 minutes or about 1-3 minutes.

Following the delivery of disinfecting fog, there may an incubation step during which there is not flow of liquid or air through the nozzles. During this time, the disinfectant may be allowed to settle and the disinfecting agent(s) allowed to chemically disinfect the internal surfaces of the equipment being treated. The incubation period can generally be in the range of 1-100 minutes such as about 1-60 minutes, such as about 1-30 minutes, such as about 1-20 minutes, about 1-15 minutes, about 1-10 minutes or about 1-5 minutes.

Following incubation with disinfectant, there can be a rinsing step, wherein water and air are delivered through the nozzle. This step serves the purpose of rinsing the treated surfaces, thereby removing the disinfectant. The rinsing step can be performed over a period of 1-100 minutes such as about 1-60 minutes, such as about 1-30 minutes, such as about 1-20 minutes, about 1-15 minutes, about 1-10 minutes or about 1-5 minutes.

Following the rinsing and treating steps, the nozzles in the system can be cleaned by driving pressurized gas only through the nozzles. This step serves to remove any debris from the nozzles, thus preventing clogging. After this step, the system is ready for the next round of rinsing/disinfection.

Any one or combinations of the foregoing steps of rinsing and disinfecting can be performed while operating the machinery being treated in the absence of food. Thereby, moving surfaces can be treated uniformly, ensuring a penetrating and thorough disinfection of the food processing equipment. This way, it is also possible to reach surfaces/spaces that otherwise could be hard to reach, and this also serves to ensure uniform cleaning and/or disinfection of the equipment.

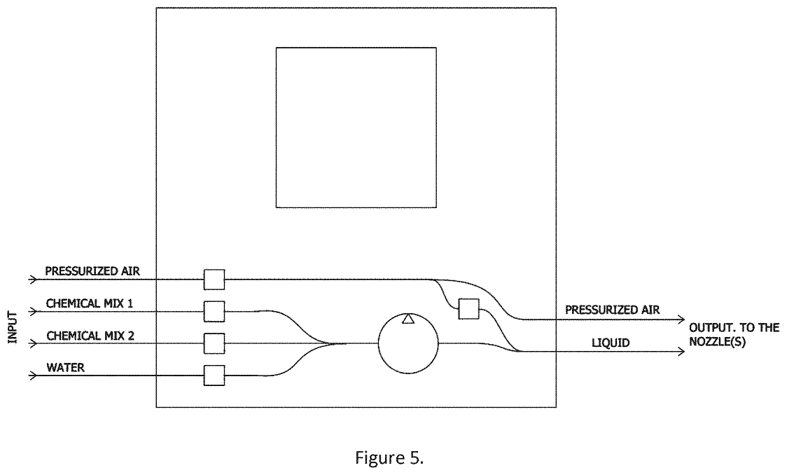

FIG. 5 shows a control and mixing unit that operates the disinfection procedures described herein through a computer or remote control. Pressurized air and liquid are delivered via separate piping to nozzles for generation of fog. The mixing unit mixes two different chemical mixes (e.g. detergents or disinfectants) and optionally also water. Square boxes on the piping indicate solenoid valves and the round ball indicates a liquid pump. These are remotely and computer controlled during disinfection. The control and mixing unit can thus (1) adjust the type and concentration of detergent/disinfectant to be delivered, (2) the pressure/rate of delivery of the liquid delivery, and (3) pressure and flow rates of the pressurized gas or air.

An advantage of the disinfecting method described herein is that there is less build-up of contamination/dirt on surfaces that have been treated with the disinfectant. Thereby conventional cleaning of the food processing equipment is made more efficient and easier. This is believed to be due to less buildup of biofilm and/or bioorganism growth on the surface of the treated equipment, to which other contaminants can easily bind or attach. The invention therefore provides important advantages beyond those of the disinfection step itself.

As will be apparent from the foregoing, the present invention provides numerous advantages over the prior art:
Improved disinfection/cleaning of food processing equipment
Reduced risk of harmful microbial contamination of food being processed
System that includes built-in nozzles for providing efficient disinfection
Less need for general cleaning due to less biofilm growth
Less use of disinfectant, thereby environmentally friendly
Reduced disinfection and cleaning cost
   As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.
   Throughout the description and claims, the terms “comprise”, “including”, “having”, and “contain” and their variations should be understood as meaning “including but not limited to” and are not intended to exclude other components.
   The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).
   It will be appreciated that variations to the foregoing embodiments of the invention can be made while still falling within the scope of the invention n. Features disclosed in the specification, unless stated otherwise, can be replaced by alternative features serving the same, equivalent or similar purpose. Thus, unless stated otherwise, each feature disclosed represents one example of a generic series of equivalent or similar features.
   Use of exemplary language, such as “for instance”, “such as”, “for example” and the like, is merely intended to better illustrate the invention and does not indicate a limitation on the scope of the invention unless so claimed. Any steps described in the specification may be performed in any order or simultaneously, unless the context clearly indicates otherwise.
   All the features and/or steps disclosed in the specification can be combined in any combination, except for combinations where at least some of the features and/or steps are mutually exclusive. In particular, preferred features of the invention are applicable to all aspects of the invention and may be used in any combination

### EXAMPLE 1

This example demonstrates the use of the invention to clean and disinfect the interior of a cutting and trimming machine processing fresh fish and how the fine fog mist spreads over the whole cabinet and contacts all walls, and also any hard-to-reach parts such as cutter knifes, cutting units and belt and therefore effectively disinfects all corners and crevices of the machine.

In Table 1 the properties and effectiveness of the present invention are compared with the state of the art of the most currently used manual methods for cleaning of cutting and trimming machines in the invention.

TABLE 1
Comparison of the disinfection efficiency of the invention
compared to current manual cleaning methods.

| Key properties in comparing disinfection | Manual | |
|---|---|---|
| methods | Cleaning | D-Tech |
| Biofilm eradication efficiency | Moderate | High |
| Suitable for difficult-to-access areas | No | Yes |
| Manual handling required | Yes | No |
| Water use per disinfection (L) | High >10 | Low ~0.8 |
| Time required (min) | ~30 | ~3 |
| Risk of run-off into environment | Yes | No |
| Biofilm as measured by ATP | High | Low |
| Bacteria on surface - cfu of E. coli | Frequently | Not |
| | detected | detected |

### EXAMPLE 2

This example demonstrates the effect of the invention when adapted for use for disinfecting a commercial cutting and trimming machine (FleXicut from Marel). This kind of machine will be cleaned and disinfected daily with fog according to the invention. The effects were measured with standard methods of measuring ATP (UltraSnap ATP surface test) and presence of E. coli as viable cells by colony forming units (Micro Snap E. coli and coliforms).

TABLE 2
Demonstration of the efficiency of the invention compared
to situation before using the invention in disinfection
of a FleXicut machine in real-life use for fish cutting.

| | Days after initial disinfection | | | |
|---|---|---|---|---|
| Position in a FleXicut machine | 0 | 5 | 10 | 20 |
| Knifes - ATP units | 181 | 85 | 24 | 0 |
| Cutting unit - ATP units | 3 | 1 | 0 | 0 |
| Belt unit - ATP units | 47 | 9 | 0 | 0 |
| Cutting unit - E. coli, cfu | 72 | 0 | 0 | 0 |
| Knife & Belts units - E. coli, cfu | 0 | 0 | 0 | 0 |

As can be seen in the table, regular (daily) use of the system leads to great reduction in cfu count after only a few days, with the count being essentially zero after 10-20 days of treatment.

### EXAMPLE 3

This example demonstrates the effect of the invention on cleaning efficiency when adapted for use for disinfecting a commercial cutting and trimming machines in regular use.

Improvements or estimated savings in manhours used for cleaning before and after
using the fog disinfection of the invention at least 4 times in regular cleaning

| Tested in type of company and food | Without using | After using disinfecting |
|---|---|---|
| processing machine | disinfecting fog | for daily for one week |
| Daily cleaning of a FleXicut | 3 hours | 2 hours |
| processing machine in whitefish | | |
| processing company | | |
| Daily cleaning of a FleXicut | 3 hours | 2 hours |
| processing machine in salmon | | |
| processing company | | |
| Weekly cleaning of Blast freezers in | 4 hours | 2 hours |
| whitefish processing company | | |
| Daily cleaning of a vacuum cyclone | Prevalent problem | Listeria no longer |
| collecting gut material in a salmon | with Listeria and | detected and no |
| processing company | frequent complaints | complaints |

The marked improvements in cleaning efficiency was a surprising effect of the use of the invention. The explanation of improved cleaning and shorter cleaning time by use of the invention is apparently because when the bacteria are eliminated from the surfaces, a biofilm can no longer build up. When the active biofilm is present it acts as a glue so the fine flesh particles circulating in the air will not stick as efficiently to the surfaces and are therefore easily removed by normal cleaning, resulting in substantial savings in the respective companies.

### REFERENCES

1. Pradhan, A K, Li M, Li Y, Kelso L C, Costello T A, Johnson M G. 2012. A modified Weibull model for growth and survival of Listeria innocua and Salmonella typhimurium in chicken breasts during refrigerated and frozen storage. Poult Sci. 91, 1482-8.
2. Moretro, T., Fanebust, H. Fagerlund, A., Langsrud, S., 2019. Whole room disinfection with hydrogen peroxide mist to control Listeria monocytogenes in food industry related environments International J. Food Microbiology 292, 118-125.
3. Morey, A, and Singh M. 2012. Low-temperature survival of Salmonella spp. in a model food system with natural microflora. Foodborne Pathog Dis. 9, 218-23.
4. Huang, L. 2010. Growth kinetics of Escherichia coli O157:H7 in mechanically-tenderized beef. Int. J. Food Microbiol. 140, 40-48.
5. Hjalmarsson, H. and Jonsson, E. B., 2010. Food processing apparatus for detecting and cutting tough tissues from food items. Patent US20120307013A1.
6. Finnsson, Th. and Hallvardsson, K., 2014. A cutting system for cutting food products. Patent application EP2957175A1.
7. Hallvardsson, K. and Gudlaugsson, H., (2014). A pin bone removal system. Patent application WO2016008926A2.
8. Blaine, G. R., J. A. Hocker, A. Steffens, 2016. Apparatus for acquiring and analyzing product-specific data for products of the food processing industry as well as a system comprising such an apparatus and a method for processing products of the food processing industry. Patent application US20180027848A1.
9. Burfoot, D., K. Hally, K. Browny and Y. Xu. 1999. Fogging for the disinfection of food processing factories and equipment Trends in Food Sci. & Technol. 10, 205-210.
10. Crosfield, R., A. Cavallo, F. Colella, R. Carvel, J. L. Torero & G. Rein, 2009. Approximate travelling distances of water mist droplets in tunnels. International Water Mist Conference, London, Sep. 23-24, 2009.
11. Chang G. and H. Chan-Myers. 2012. Comparison of Surface Disinfection Capabilities of Two Peroxide Based Disinfectants in an Automated Fogging Machine in a 72 m3 Room. Am. J. Infection Control, 40, e38-e39
12. Agmont E Silva, C. V. 2017. System, method and process for disinfection of internal surfaces in aseptic tanks and pipelines by flooding with sanitizing fog. Patent application US20170173199A1

1. A system for the disinfection of food processing equipment having means for input, processing and/or output of food parts, the system comprising one or more nozzle for delivering disinfecting fog;
piping means for delivering gas and disinfectant liquid from respective sources of gas and disinfectant liquid to the one or more nozzle;
at least one gas flow rate controller and at least one liquid flow controller, for controlling gas and liquid flow rates in the one or more nozzle;
wherein the one or more nozzle is adapted to be positioned on food processing equipment so that during use, disinfecting fog delivered by the at least one nozzle is directed towards at least one internal surface of the food processing equipment, wherein free unobstructed space in front of the nozzle, for the expansion of the sanitizing fog towards the at least one internal surface, is at least 20 cm, and
wherein the nozzle is adapted to deliver sanitizing fog that expands from the nozzle at an angle of at least 20° relative to the direction of the expansion.

2-4. (canceled)

5. The system of claim 1, wherein the one or more nozzle is adapted so that the disinfecting fog droplets delivered by the nozzle have a diameter that is smaller than 20 μm.

6-7. (canceled)

8. The system according to claim 1, wherein the system comprises mixing means to generate a ready-to-use liquid disinfectant by mixing water and one or more concentrated disinfectant solution.

9. The system of claim 1, further comprising one or more disinfectant supply, for supplying the disinfectant liquid to the one or more nozzle.

10-11. (canceled)

12. The system of claim 1, wherein the liquid disinfectant comprises one or more active substances selected from isopropyl alcohol and dodecyldimethylammonium chloride, benzalkonium chloride, and Poly(hexamethylene biguanide) hydrochloride.

13. The system according to claim 1, wherein the liquid disinfectant comprises one or more active substances selected from isopropyl alcohol and dodecyldimethylammonium chloride, benzalkonium chloride, and Poly(hexamethylene biguanide) hydrochloride, and the liquid disinfectant further comprising one or more additional active disinfecting substances such as Peroxyacetic acid, Peracetic acid and Ethaneperoxoic acid.

14-15. (canceled)

16. The system according to claim 1, wherein the food processing equipment comprises, or consists of, high precision cutting and/or trimming equipment for fish, poultry and/or meat.

17. A food processing system having internal disinfecting and/or cleaning means, the system comprising:
at least one food processing unit, wherein the at least one food processing unit comprises a housing and food processing means arranged within said housing; and
a plurality of nozzles arranged on an inside surface of said housing, the nozzles being adapted to deliver a stream of a disinfectant and/or cleaning fluid towards the food processing means;
wherein said plurality of nozzles is arranged on an internal surface of said housing such that unobstructed space between the nozzles and food processing means within said housing, in the direction of delivery of a stream of disinfectant from the nozzles, is at least 20 cm.

18. The food processing system of claim 17, wherein the nozzles are adapted such that the stream of disinfectant can expand from the nozzle at an angle of at least 20° relative to the direction of the expansion.

19-20. (canceled)

21. The system of claim 17, wherein the food processing means comprises means for high precision cutting and/or trimming of food items, in particular fish, poultry or meat.

22. The system of claim 17, wherein the nozzles are adapted to deliver a stream of a disinfecting fog, the fog comprising droplets having a diameter that is smaller than 20 μm, preferably a diameter smaller than 10 μm, more preferably a diameter smaller than 5 μm.

23-27. (canceled)

28. The system of claim 17, wherein the system further comprises a mixing unit, for generating a ready-to-use liquid disinfectant by mixing water and one or more concentrated disinfectant solution.

29-31. (canceled)

32. A method of disinfecting food processing equipment having a housing and internal means for input, processing and/or output of food parts comprised within said housing, the method comprising introducing a disinfecting fog containing droplets that are smaller than 20 μm in diameter into the food processing equipment, so as to flood internal surfaces of the food processing equipment with the disinfectant, wherein the introducing is performed by delivering disinfecting fog via one or more nozzle that is positioned on an internal surface of the housing.

33. The method of claim 32, wherein the step of introducing a disinfecting fog is preceded by a pretreatment step, wherein a fog containing aqueous droplets that are smaller than 20 μm in diameter and do not contain disinfectant is introduced into the food processing equipment.

34. The method of claim 32, wherein the step of introducing a disinfecting fog is followed by at least one step of rinsing by introduction of a fog containing aqueous droplets that are smaller than 20 μm in diameter and do not contain disinfectant is introduced into the food processing equipment.

35. The method of claim 33 wherein the droplets that do not contain disinfectant are water droplets

36. The method of claim 32, wherein the one or more nozzle is directed towards at least one internal surface of the food processing equipment such that free unobstructed space in front of the nozzle, for the expansion of the sanitizing fog towards the internal means for input, processing and/or output of food parts is at least 20 cm.

37. The method of claim 32, wherein the disinfecting fog is delivered by expansion from the nozzle, at an angle of at least 20° relative to the direction of the expansion.

38. The method of claim 32, wherein fog is delivered through the nozzles while simultaneously operating the food processing equipment in the absence of food.

39-41. (canceled)

42. The method of claim 32, wherein the method is performed using a system comprising:
one or more nozzle for delivering disinfecting fog;
piping means for delivering gas and disinfectant liquid from respective sources of pas and disinfectant liquid to the one or more nozzle;
at least one gas flow rate controller and at least one liquid flow controller, for controlling gas and liquid flow rates in the one or more nozzle,
wherein the one or more nozzle is adapted to be positioned on food processing equipment so that during use, disinfecting fog delivered by the at least one nozzle is directed towards at least one internal surface of the food processing equipment, wherein free unobstructed space in front of the nozzle, for the expansion of the sanitizing fog towards the at least one internal surface, is at least 20 cm, and
wherein the nozzle is adapted to deliver sanitizing fog that expands from the nozzle at an angle of at least 20° relative to the direction of the expansion.
